(19) United States
(12) Patent Application Publication
(10) Pub. No.: US 2025/0319084 A1
(43) Pub. Date: Oct. 16, 2025

(54) ENDOCRINE THERAPY AND ABEMACICLIB COMBINATION FOR THE ADJUVANT TREATMENT OF NODE-POSITIVE, EARLY STAGE, HORMONE RECEPTOR-POSITIVE, HUMAN EPIDERMAL GROWTH FACTOR RECEPTOR 2-NEGATIVE BREAST CANCER

(71) Applicant: Eli Lilly and Company, Indianapolis, IN (US)

(72) Inventor: Ian Charles SMITH, Carmel, IN (US)

(21) Appl. No.: 19/203,562

(22) Filed: May 9, 2025

Related U.S. Application Data
(63) Continuation of application No. 17/462,872, filed on Aug. 31, 2021.
Application No. 17/462,872 is a continuation of application No. 16/605,226, filed on Oct. 15, 2019, now abandoned.
(60) Provisional application No. 62/500,068, filed on May 2, 2017.

(51) Int. Cl. A61K 31/506; A61K 31/138; A61K 31/4196; A61K 31/5685; A61P 35/04

(52) U.S. Cl. CPC A61K 31/506; A61K 31/138; A61K 31/4196; A61K 31/5685; A61P 35/04

(57) ABSTRACT

The present invention discloses an adjuvant treatment of node-positive, carly stage, hormone receptor-positive (HR+), human epidermal growth factor receptor 2-negative (HER2—) breast cancer comprising administering an effective amount of an endocrine therapy in combination with an effective amount of abemaciclib or a pharmaceutically acceptable salt thereof.

The present invention relates to the field of adjuvant treatment of node-positive, early stage, hormone receptor-positive (HR+), human epidermal growth factor receptor 2-negative (HER2−) breast cancer.

The currently approved standards of care offered to patients with node-positive, early stage, hormone receptor-positive (HR+), human epidermal growth factor receptor 2-negative (HER2−) breast cancer are adjuvant cytotoxic chemotherapy and endocrine therapy. Unfortunately, with current standard of care adjuvant therapy, approximately 30% of women with hormone receptor positive (HR+) breast cancer initially diagnosed with early stage disease experience distant relapse with metastases. Consequently, there is a critical need for more optimal adjuvant therapy in patients with early HR+breast cancer who have a high likelihood of distant recurrence.

According to one aspect of the present invention, there is presented a method of providing adjuvant treatment to a patient with a prior diagnosis of node-positive, early stage, HR+, HER2− breast cancer which has been resected comprising administering an effective amount of an endocrine therapy in combination with an effective amount of abemaciclib or a pharmaceutically acceptable salt thereof for a time period sufficient to increase distant relapse-free survival. Preferably the endocrine therapy is tamoxifen or a pharmaceutically acceptable salt thereof. Preferable the endocrine therapy is letrozole. Preferably the endocrine therapy is anastrozole. Preferably the endocrine therapy is exemestane.

According to another aspect of the present invention, there is presented a combination comprising therapeutically effective amounts of abemaciclib, or a pharmaceutically acceptable salt thereof, and an endocrine therapy for simultaneous, separate, or sequential use in providing adjuvant treatment to a patient with a prior diagnosis of node-positive, early stage, HR+, HER2− breast cancer which has been resected, wherein administration of the abemaciclib or the salt thereof and the endocrine therapy is for a time period sufficient to increase distant relapse-free survival. Preferably the endocrine therapy is tamoxifen or a pharmaceutically acceptable salt thereof. Preferable the endocrine therapy is letrozole. Preferably the endocrine therapy is anastrozole. Preferably the endocrine therapy is exemestane.

According to another aspect of the present invention, there is presented a therapeutically effective amount of abemaciclib, or a pharmaceutically acceptable salt thereof, for use in simultaneous, separate, or sequential combination with a therapeutically effective amount of an endocrine therapy in providing adjuvant treatment to a patient with a prior diagnosis of node-positive, early stage, HR+, HER2− breast cancer which has been resected, wherein administration of the abemaciclib or the salt thereof and the endocrine therapy is for a time period sufficient to increase distant relapse-free survival. According to another aspect of the present invention, there is presented a therapeutically effective amount of an endocrine therapy for use in simultaneous, separate, or sequential combination with a therapeutically effective amount of abemaciclib, or a pharmaceutically acceptable salt thereof, in providing adjuvant treatment to a patient with a prior diagnosis of node-positive, early stage, HR+, HER2− breast cancer which has been resected, wherein administration of the abemaciclib or the salt thereof and the endocrine therapy is for a time period sufficient to increase distant relapse-free survival. Preferably the endocrine therapy is tamoxifen or a pharmaceutically acceptable salt thereof. Preferable the endocrine therapy is letrozole. Preferably the endocrine therapy is anastrozole. Preferably the endocrine therapy is exemestane.

According to another aspect of the present invention, there is also presented the use of abemaciclib, or a pharmaceutically salt thereof, in the manufacture of a medicament for providing adjuvant treatment of node-positive, early stage, HR+, HER2− breast cancer which has been resected wherein the abemaciclib, or the salt thereof, is to be administered in simultaneous, separate, or sequential combination with an endocrine therapy for a time period sufficient to increase distant relapse-free survival. Preferably the endocrine therapy is tamoxifen or a pharmaceutically acceptable salt thereof. Preferable the endocrine therapy is letrozole. Preferably the endocrine therapy is anastrozole. Preferably the endocrine therapy is exemestane.

According to another aspect of the present invention, there is also presented the use of an endocrine therapy in the manufacture of a medicament for providing adjuvant treatment of node-positive, early stage, HR+, HER2− breast cancer which has been resected wherein the endocrine therapy is to be administered in simultaneous, separate, or sequential combination with abemaciclib, or a pharmaceutically salt thereof, for a time period sufficient to increase distant relapse-free survival. Preferably the endocrine therapy is tamoxifen or a pharmaceutically acceptable salt thereof. Preferable the endocrine therapy is letrozole. Preferably the endocrine therapy is anastrozole. Preferably the endocrine therapy is exemestane.

For all of the preceding aspects, the following are preferred dosing. Preferably, abemaciclib, or the pharmaceutically salt thereof, is administered at a dose of 50 mg to 200 mg twice a day. Also preferably, abemaciclib, or the pharmaceutically salt thereof, is administered at a dose of 100 mg to 150 mg twice a day. More preferably, abemaciclib, or the pharmaceutically salt thereof, is administered at a dose of 200 mg twice a day. More preferably, abemaciclib, or the pharmaceutically salt thereof, is administered at a dose of 150 mg twice a day in a 28-day cycle. More preferably, abemaciclib, or the pharmaceutically salt thereof, is administered at a dose of 100 mg twice a day in a 28-day cycle. More preferably, abemaciclib, or the pharmaceutically salt thereof, is administered at a dose of 50 mg twice a day in a 28-day cycle. Preferably, abemaciclib is administered orally. More preferably, abemaciclib is administered by capsule. Also more preferably, abemaciclib is administered by tablet.

Preferably, an endocrine therapy is administered as described on the approved label of the particular endocrine therapy. For example, tamoxifen or a pharmaceutically acceptable salt thereof may be administered at 20-40 mg/day. For doses over 20 mg, the dose should be administered in a divided dose of morning and evening. Doses are preferably oral. For example, anastrazole may be administered at 1 mg/day. Doses are preferably oral. For example, letrozole may be administered at 2.5 mg/day. Doses are preferably oral. For example, exemestane may be administered at 25 mg/day. Doses are preferably oral.

Preferably, tamoxifen or a pharmaceutically acceptable salt thereof is administered at 20-40 mg/day and abemaciclib or a pharmaceuticeutically acceptable salt thereof is administered at 200 mg twice daily. Preferably, tamoxifen or a pharmaceutically acceptable salt thereof is administered at 20-40 mg/day and abemaciclib or a pharmaceuticeutically acceptable salt thereof is administered at 150 mg twice daily.

Preferably, tamoxifen or a pharmaceutically acceptable salt thereof is administered at 20-40 mg/day and abemaciclib or a pharmaceuticeutically acceptable salt thereof is administered at 100 mg twice daily. For doses of tamoxifen or a salt thereof over 20 mg, the dose should be administered in a divided dose of morning and evening. Doses are preferably oral for both tamoxifen or the salt thereof and abemaciclib or the salt thereof.

Preferably, anastrazole is administered at 1 mg/day and abemaciclib or a pharmaceutically acceptable salt therof is administered at 200 mg twice daily. Preferably, anastrazole is administered at 1 mg/day and abemaciclib or a pharmaceutically acceptable salt therof is administered at 150 mg twice daily. Preferably, anastrazole is administered at 1 mg/day and abemaciclib or a pharmaceutically acceptable salt therof is administered at 100 mg twice daily. Doses are preferably oral for both anastrazole and abemaciclib or the salt thereof.

Preferably, letrozole is administered at 2.5 mg/day and abemaciclib or a pharmaceutically acceptable salt therof is administered at 200 mg twice daily. Preferably, letrozole is administered at 2.5 mg/day and abemaciclib or a pharmaceutically acceptable salt therof is administered at 150 mg twice daily. Preferably, letrozole is administered at 2.5 mg/day and abemaciclib or a pharmaceutically acceptable salt therof is administered at 100 mg twice daily. Doses are preferably oral for both anastrazole and abemaciclib or the salt thereof.

Preferably, exemestane is administered at 25 mg/day and abemaciclib or a pharmaceutically acceptable salt therof is administered at 200 mg twice daily. Preferably, exemestane is administered at 25 mg/day and abemaciclib or a pharmaceutically acceptable salt therof is administered at 150 mg twice daily. Preferably, exemestane is administered at 25 mg/day and abemaciclib or a pharmaceutically acceptable salt therof is administered at 100 mg twice daily. Doses are preferably oral for both anastrazole and abemaciclib or the salt thereof.

As used herein, the term “patient” refers to a human.

As used herein, the terms “cancer” and “cancerous” refer to or describe the physiological condition in patients that is typically characterized by unregulated cell proliferation.

As used herein, the term “effective amount” refers to the amount or dose of abemaciclib, or a pharmaceutically acceptable salt thereof, and the amount or dose of an endocrine therapy which provides an effective response in the patient under diagnosis or treatment.

As used herein, the term “effective response” of a patient or a patient's “responsiveness” to treatment with a combination of agents refers to the clinical or therapeutic benefit imparted to a patient upon administration of abemaciclib, or a pharmaceutically acceptable salt thereof, and an endocrine therapy.

As used herein, the term “in combination with” refers to the administration of abemaciclib, or a pharmaceutically acceptable salt thereof, and an endocrine therapy either simultaneously or sequentially in any order, such as for example, at repeated intervals as during a standard course of treatment for a single cycle or more than one cycle, such that one agent can be administered prior to, at the same time, or subsequent to the administration of the other agent, or any combination thereof.

Abemaciclib (LY2835219), [5-(4-ethyl-piperazin-1-ylmethyl)-pyridin-2-yl]-[5-fluoro-4-(7-fluoro-3-isopropyl-2-methyl-3H-benzoimidazol-5-yl)-pyrimidin-2-yl]-amine, is a CDK inhibitor that targets the CDK4 and CDK6 cell cycle pathway, with antineoplastic activities. Abemaciclib, including salt forms, and methods of making and using this compound including for the treatment of cancer, in particular, breast cancer are disclosed in WO2010/075074. Abemaciclib has the following structure:

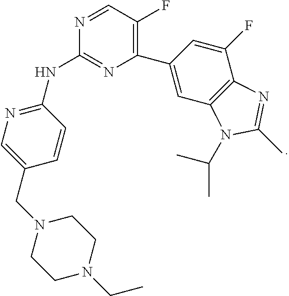 [SMILES: CCN1CCN(Cc2ccc(Nc3ncc(F)c(-c4cc(F)c5nc(C)n(C(C)C)c5c4)n3)nc2)CC1]

Abemaciclib showed antitumor activity within multiple preclinical pharmacology models and an acceptable toxicity profile in nonclinical species. Abemaciclib has been shown to significantly inhibit tumor growth in multiple murine xenograft models for human cancer, including breast cancer. Growth inhibition in vitro across a diverse panel of 46 breast cancer cell lines, representing the known molecular subgroups of breast cancer, indicates that sensitivity to CDK4 and CDK6 inhibition is greater in ER+breast cancers with luminal histology.

Continuously dosed abemaciclib has demonstrated robust single-agent clinical activity in both the JPBA Phase 1b study with a response rate of 33.3% across dose levels and the MONARCH 1 Phase 2 study with a response rate of 19.7% (at 200-mg dose twice daily), in patients with heavily pretreated HR+metastatic breast cancer that is refractory to endocrine therapy (Patnaik et al. 2016; Dickler et al. 2016).

When combined with standard endocrine therapies (for example, tamoxifen, letrozole, anastrozole, exemestane), abemaciclib demonstrated early evidence of antitumor activity against HR+mBC (objective response rate: 10% to 40%; disease control rate: 60% to 87.5%) and was tolerable in the ongoing JPBH Phase 1b study (Beeram et al. 2016). In JPBH, the most common adverse events (AEs) experienced by patients receiving abemaciclib plus endocrine therapy included fatigue, nausea, diarrhea, leukopenia, lymphopenia, neutropenia, and anemia, which are predominantly of low-grade severity and appear to be dose-dependent.

In early neoadjuvant breast cancer setting, the ongoing neoMONARCH Phase 2 open-label,randomized study (13Y-MC-JPBY) showed an acceptable safety profile for abemaciclib (150 mg twice daily) as monotherapy and in combination with anastrozole, with reduction of breast cancer tumor cell proliferation marker (Ki67 index) to a significantly greater extent than anastrozole alone (Hurvitz et al. 2016).

As used herein, the term “endocrine therapy” means tamoxifen or a pharmaceutically acceptable salt thereof, anastrozole, letrozole, or exemestane.

Tamoxifen is a selective estrogen receptor modulators (SERM) with tissue-specific activities for the treatment and prevention of estrogen receptor positive breast cancer. Tamoxifen acts as an anti-estrogen (inhibiting agent) in the mammary tissue, but as an estrogen (stimulating agent) in cholesterol metabolism, bone density, and cell proliferation in the endometrium. Tamoxifen has the following structure:

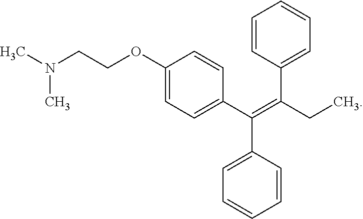 [SMILES: CC/C(=C(\c1ccccc1)c1ccc(OCCN(C)C)cc1)c1ccccc1]

Anastrozole is a drug indicated in the treatment of breast cancer in post-menopausal women. It is used both in adjuvant therapy (i.e. following surgery) and in metastatic breast cancer. It decreases the amount of estrogens that the body makes.

Anastrozole belongs in the class of drugs known as aromatase inhibitors. It inhibits the enzyme aromatase, which is responsible for converting androgens (produced by women in the adrenal glands) to estrogens. Anastrozole has the following structure:

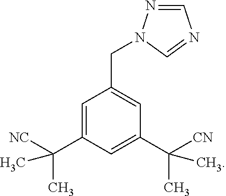 [SMILES: CC(C)(C#N)c1cc(Cn2cncn2)cc(C(C)(C)C#N)c1]

Letrozole is an oral non-steroidal aromatase inhibitor that has been introduced for the adjuvant treatment of hormonally-responsive breast cancer. Letrozole has the following structure:

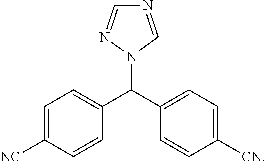 [SMILES: N#Cc1ccc(C(c2ccc(C#N)cc2)n2cncn2)cc1]

Exemestane is an oral steroidal aromatase inhibitor used in the adjuvant treatment of hormonally-responsive (also called hormone-receptor-positive, estrogen-responsive) breast cancer in postmenopausal women. It acts as a false substrate for the aromatase enzyme, and is processed to an intermediate that binds irreversibly to the active site of the enzyme causing its inactivation. Exemestane has the following structure:

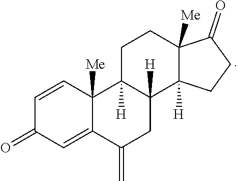

As used herein, the term “resection” means surgical removal of malignant tissue characteristic of breast cancer from a patient. According to one embodiment, resection means removal of malignant tissue such that the presence of remaining malignant tissue within said patient is undetectable with available methods. According to another embodiment of the invention resection means removal of breast cancer such that the presence of remaining breast cancer with said patient is undetectable.

As used herein, the term “distant relapse-free survival” means the time from starting treatment of the combination of abemaciclib or the salt thereof and the endocrine therapy to disease recurrence, distant metastases, or death from any cause.

As used herein, the term “early stage” means cancers that may have spread to nearby lymph nodes but not to distant parts of the body.

As used herein, the term “treating”, “treatment”, or adjuvant treatment” means the administration of a drug or drugs to a patient after surgical resection of one or more cancerous tumors, where all detectable and resectable disease (for example, cancer) has been removed from the patient, but where there remains a statistical risk of relapse due to occult disease, for the purpose of diminishing the likelihood or the severity of reoccurance of the disease, or to delay the onset of the biological manifestation of the reoccurance of the disease.

“Ki67 antigen” or simply “Ki67” (also known as antigen identified by monoclonal antibody Ki-67) means a nuclear protein encoded by the MKI67 gene that is expressed in all phases of the cell cycle other than the G0 phase and has been reported as an independent prognostic factor in early breast cancer (Dowsett et al. 2011). In HR+ breast cancer, patients with high levels of Ki67 have been shown to have higher disease recurrence rates while receiving adjuvant endocrine therapy following surgery. In the BIG 1-98 study (Viale et al. 2008), patients receiving letrozole with HR+ early breast cancer involving their axillary lymph nodes and low (≤11%) Ki67 levels at baseline had a 4-year disease-free survival of 93% compared to 85% for patients with higher Ki67 values (>11%). Currently, there is no consensus as to the precise baseline level of Ki67 that would differentiate a patient for being of higher or lower risk of disease recurrence whilst on adjuvant endocrine therapy. However, the majority of the panel of the St Gallen International Expert Consensus on the Primary Therapy of Early Breast Cancer 2015 was prepared to accept a threshold value of Ki67 within the range of 20% to 29% (Vasconcelos et al. 2016) as indicative of high-risk group appropriate to receive adjuvant chemotherapy.

### EXAMPLE 1

### A Randomized, Open-Label, Phase 3 Study of Abemaciclib Combined With Standard Adjuvant Endocrine Therapy Versus Standard Adjuvant Endocrine Therapy Alone in Patients With High Risk, Node Positive, Early Stage, Hormone Receptor Positive, Human Epidermal Receptor 2 Negative, Breast Cancer

The primary objective of this study is to evaluate the efficacy, in terms of invasive disease-free survival (IDFS), as defined by the STEEP System, for patients with HR+, HER2− early stage breast cancer for abemaciclib 150 mg twice daily plus adjuvant endocrine therapy versus adjuvant endocrine therapy alone.

The secondary objectives of this study is to
evaluate the efficacy, in terms of IDFS, for patients with HR+, HER2− early stage breast cancer with Ki67 index ≥20% (both Cohort 1 and Cohort 2 by central lab);
evaluate the efficacy of abemaciclib plus adjuvant endocrine therapy versus adjuvant endocrine therapy alone in terms of distant relapse-free survival (DRFS) and overall survival (OS);
assess the safety profile of abemaciclib plus adjuvant endocrine therapy compared to adjuvant endocrine therapy alone;
evaluate the relationship between abemaciclib exposure and clinical (efficacy and safety) outcomes;
evaluate abemaciclib plus adjuvant endocrine therapy, versus adjuvant endocrine therapy alone, in terms of general oncology and breast cancer self-reported health-related quality of life (Functional Assessment of Cancer Therapy [FACT]-Breast 37-item questionnaire), endocrine therapy-specific symptoms (Functional Assessment of Cancer Therapy-Endocrine Symptoms (Version 4) [FACT-ES] 19-item subscale and 2 Functional Assessment of Chronic Illness Therapy Item Library [FACIT] (Version 2) sourced items of cognitive symptoms and 3 FACIT-sourced items for bladder symptoms), and fatigue experienced during abemaciclib and/or endocrine therapy (FACIT-Fatigue 13-item subscale); and
evaluate health status to inform decision modeling for health economic evaluation using the EuroQol five-dimension five-level questionnaire.

The study will screen approximately 4200 patients, and approximately 3580 patients will be enrolled and subdivided into 2 cohorts: those eligible based on nodal status, tumor size, or grade regardless of Ki67 status, Cohort 1, and those with at least 1 positive node and eligible exclusively based on a Ki67 status, Cohort 2 (that is, those patients not eligible based on tumor size or grade). Cohort 1 will enroll approximately 3080 patients and Cohort 2 will enroll approximately 500 patients.

Patients in both treatment arms will receive standard adjuvant endocrine therapy of physician's choice (such as tamoxifen or an aromatase inhibitor, with or without ovarian function suppression per standard practice). Patients in both arms may have started standard adjuvant endocrine therapy within 8 weeks prior to randomization, and the same or another endocrine therapy will be continued during the course of the study and in the absence of disease recurrence. Consistent with standard guidelines, aromatase inhibitor should be at least part of endocrine therapy for postmenopausal patients. Adjuvant treatment with fulvestrant is not allowed at any time during the study. Randomization must occur no more than 12 weeks after completion of last non-endocrine therapy (surgery, or chemotherapy or radiotherapy). Patients randomized to the experimental arm will receive abemaciclib orally at 150 mg twice daily for up to 2 years or until evidence of disease recurrence or other discontinuation criteria are met, whichever occurs first. Endocrine therapy will be taken as prescribed during the on-study treatment period. (Years 1 and 2). In Year 3 and beyond, standard adjuvant endocrine therapy will continue to complete at least 5 years per investigator's discretion as part of standard of care.

We claim:

1. A method of providing adjuvant treatment to a patient with a prior diagnosis of node-positive, early stage, HR+, HER2− breast cancer which has been resected comprising administering an effective amount of an endocrine therapy in combination with an effective amount of abemaciclib or a pharmaceutically acceptable salt thereof for a time period sufficient to increase distant relapse-free survival.

2. The method according to claim 1 wherein the endocrine therapy is tamoxifen or a pharmaceutically acceptable salt thereof.

3. The method according to claim 1 wherein the endocrine therapy is letrozole.

4. The method according to claim 1 wherein the endocrine therapy is anastrozole.

5. The method according to claim 1 wherein the endocrine therapy is exemestane.

6. The method according to claim 1 wherein the abemaciclib or the salt thereof is administered at 150 mg twice daily.

7. The method according to claim 1 wherein the abemaciclib or the salt thereof is administered at 100 mg twice daily.

8.-16. (canceled)
